(19) United States
(12) Patent Application Publication
(10) Pub. No.: US 2023/0149930 A1
(43) Pub. Date: May 18, 2023

(54) TOXICITY EVALUATION SYSTEM AND METHOD FOR TOXICOLOGICAL EVALUATION USING THE SAME

(71) Applicant: KYUNGPOOK NATIONAL UNIVERSITY INDUSTRY-ACADEMIC COOPERATION FOUNDATION, Daegu (KR)

(72) Inventors: Sung Wook NAM, Daegu (KR); Mae Ja Park, Daegu (KR)

(21) Appl. No.: 17/985,129

(22) Filed: Nov. 10, 2022

(30) Foreign Application Priority Data
Nov. 15, 2021 (KR) 10-2021-0157039

(51) Int. Cl. B01L 3/00; C12M 3/00; C12M 3/06

(52) U.S. Cl. CPC B01L 3/502761; C12M 21/06; C12M 23/16; B01L 2400/0487; B01L 2200/0647; B01L 2300/0816

(57) ABSTRACT

Disclosed is a toxicity evaluation system including a chip including a passage, in which a sample flows, and a plurality of recesses provided on the passage, and that traps objects included in the sample, and a pump that injects the sample into the chip. Each of the plurality of recesses has a size, by which one spawn included in the sample is trapped.

### STATEMENT REGARDING PRIOR DISCLOSURES BY THE INVENTORS

The inventors of the present application authored and disclosed the subject matter of the present application on Sep. 10, 2021. This prior disclosures has been submitted in an Information Disclosure Statement in the present application as “NAM, Sung-Wook et al. “Xenopus chip for single-egg trapping, in vitro fertilization, development, and tadpole escape.” Biochemical and Biophysical Research Communications 569 (2021) 29-34. Sep. 10, 2021. English Language. 6 pages.”

### CROSS-REFERENCE TO RELATED APPLICATIONS

A claim for priority under 35 U.S.C. § 119 is made to Korean Patent Application No. 10-2021-0157039 filed on Nov. 15, 2021, in the Korean Intellectual Property Office, the entire contents of which are hereby incorporated by reference.

### BACKGROUND

Embodiments of the inventive concept described herein relate to a toxicity evaluation system, and a method for a toxicological evaluation using the same. In more detail, the inventive concept relates to an invention regarding a system including a chip that allows in vitro fertilization, development of embryos, and toxicological evaluation of spawns of a South African Xenopus laevis having claws, which is one of vertebrates.

A Xenopus laevis is an animal that does in vitro fertilization, and it is easy to observe a generation process outside the body. 678 studies supported by National Institutes of Health (NIH) in 2010 use the Xenopus laevis as an experiment animal, and about 217,000,000 dollars (about 2 trillion Won) are supported. South African Xenopus laevis having claws is widely used as an animal model for studying development of vertebrates. In particular, because a development process that continues after fertilization of oocytes and sperms of the Xenopus laevis may be observed outside the body, it corresponds to an important experiment animal in studying an initial development step of the vertebrates.

### SUMMARY

Embodiments of the inventive concept provide an evaluation system, by which an initial development step of vertebrates may be easily observed.

The problems that are to be solved by the inventive concept are not limited to the above-mentioned problems, and the unmentioned problems will be clearly understood by those skilled in the art to which the inventive concept pertains from the specification and the accompanying drawings.

A toxicity evaluation system according to an embodiment of the inventive concept is disclosed.

The toxicity evaluation system includes a chip including a passage, in which a sample flows, and a plurality of recesses provided on the passage, and that traps objects included in the sample, and a pump that injects the sample into the chip.

According to an example, each of the plurality of recesses may have a size, by which one spawn included in the sample is trapped.

According to an example, an inlet port and an outlet port may be formed at one end and an opposite end of the passage.

According to an example, the passage may be a combination of a first passage having a first width and a second passage having a second width, the first width may have a size, by which the objects included in the sample flow, and the second width may have a size, by which the objects included in the sample is prevented from flowing.

According to an example, the recesses may be provided in a direction that faces the outlet port from the inlet port.

According to an example, the pump may cause the sample to flow in any one of the first direction or the second direction.

According to an example, the sample may be any one of a solution containing the spawns, a solution containing sperms that fertilize the spawns, or a pharmaceutical sample, by which a toxicity is evaluated.

A method for performing a toxicological evaluation by using the toxicity evaluation system according to another embodiment of the inventive concept is disclosed.

The method includes trapping the spawns in the plurality of recesses by injecting the solution containing the spawns into the chip, and inducing in vitro fertilization by injecting the solution containing the sperms that fertilize the spawns into the chip.

According to an example, the method may further include injecting a sample for evaluating a toxicity of an organism, on which the in vitro fertilization has been completed, into the passage.

According to an example, the method may further include trapping the organism, on which the in vitro fertilization has been completed, by the recesses, by adjusting the pump such that the sample flows in a first direction.

According to an example, the method may further include causing the organism, on which the in vitro fertilization has been completed, to escape, by adjusting the pump such that the sample flows in a second direction.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view illustrating an example of a toxicity evaluation system according to an embodiment of the inventive concept;

FIG. 2 is a view illustrating a chip according to an embodiment of the inventive concept;

FIGS. 3A-3D is a view illustrating a process of manufacturing a chip according to FIG. 2;

FIGS. 4A-4D is a view illustrating that spawns are trapped by using a chip according to FIG. 3;

FIGS. 5A-5E is a view illustrating that an embryo is sequentially grown by using a chip according to the inventive concept;

FIGS. 6A-6H is a view illustrating that trapping or escaping of a tadpole may be adjusted by adjusting a direction of a pump; and

FIG. 7 is a flowchart illustrating a method for a toxicological evaluation according to an example of the inventive concept.

### DETAILED DESCRIPTION

The terms used in the specification and the accompanying drawings are provided to easily describe the inventive concept, and the inventive concept is not limited by the terms and the drawings.

A detailed description of, among the technologies used in the inventive concept, known technologies that are not closely relevant to the spirit of the inventive concept will be omitted.

Hereinafter, exemplary embodiments of the inventive concept will be described in detail with reference to the accompanying drawings so that those skilled in the art to which the inventive concept pertains may easily carry out the inventive concept. However, the inventive concept may be implemented in various different forms, and is not limited to the embodiments. Furthermore, in a detailed description of the preferred embodiment of the inventive concept, a detailed description of related known functions or configurations will be omitted when they may make the essence of the inventive concept unclear. In addition, the same reference numerals are used for parts that perform similar functions and operations throughout the drawings.

The expression of ‘including’ some elements may mean that another element may be further included without being excluded unless there is a particularly contradictory description. In detail, the terms “including” and “having” are used to designate that the features, the numbers, the steps, the operations, the elements, the parts, or combination thereof described in the specification are present, and may be understood that one or more other features, numbers, step, operations, elements, parts, or combinations thereof may be added.

The terms such as first and second may be used to describe various elements, but the elements are not limited to the terms. The terms may be used only for distinguishing one component from other components. For example, without departing the scope of the inventive concept, a first element may be referred to as a second element, and similarly, a second element may be referred to as a first element.

The terms of a singular form may include plural forms unless otherwise specified. Furthermore, in the drawings, the shapes and sizes of the elements may be exaggerated for clearer description.

The term ‘unit’ or ‘module’ used in the entire specification is a unit for processing at least one function or operation, and for example, may refer to a hardware element such as an FPGA or an ASIC. However, the ‘unit’ or the ‘module’ is not limited to software or hardware. The ‘unit’ and “module” may be constituted in a storage medium that may perform addressing, and may be configured to reproduce one or more processors.

Accordingly, as an example, the ‘unit’ and ‘the module’ may include elements such as software elements, object-oriented software elements, class elements, and task elements, processes, functions, attributes, procedures, sub-routines, segments of a program code, drivers, firmware, micro-codes, circuits, data, a database, data structures, tables, arrays, and parameters. The functions provided by the elements and the ‘units’ and the ‘modules’ may be separately performed by a plurality of elements and ‘units’, and may be integrated with other additional elements, and ‘the unit’ and ‘the module’.

The inventive concept discloses a Xenopus laevis chip 10 for evaluating external fertilization of a vertebrate, development of an embryo, and a development toxicity. The chip 10 according to the inventive concept may induce fertilization and embryogenesis after trapping an embryo of a single entity level in a 3D chip, and may evaluate a toxicity of a medicine. The chip 10 of the inventive concept may function as an incubator that significantly increases a probability of fertilization in a process of performing external fertilization of eyes or embryos. Furthermore, the inventive concept may supply sperms, therapeutic substances, or biomolecules at a uniform velocity through a fluid circulation system using a pump 20, and may cause continuous flows and circulations. Furthermore, after an embryo is grown up to a tadpole, the tadpole may be extracted out of a 3D chip reversely.

Hereinafter, a toxicity evaluation system 1 according to the inventive concept will be described in detail with reference to the accompanying drawings.

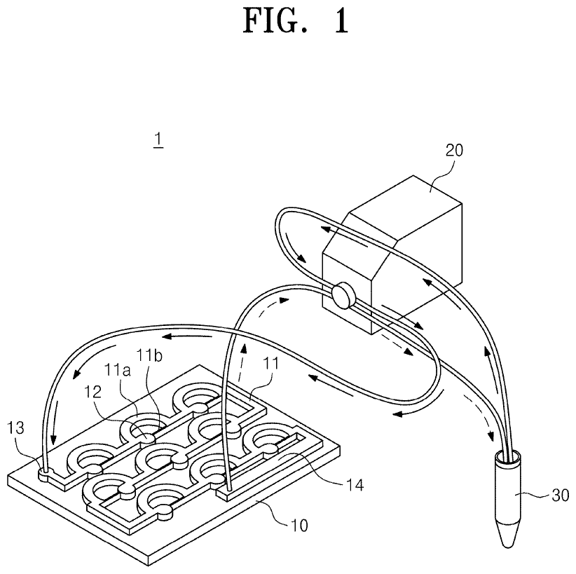

FIG. 1 is a view illustrating an example of the toxicity evaluation system 1 according to an embodiment of the inventive concept.

The toxicity evaluation system 1 according to the inventive concept may include the chip 10 including a passage 11, in which the sample may flow, and a plurality of recesses 12 provided on the passage 11, and by which objects included in the sample may be trapped. The toxicity evaluation system 1 according to the inventive concept may include the pump 20 that may inject the sample into the chip 10. Hereinafter, the chip 10 and the Xenopus laevis chip 10 may have the same meaning.

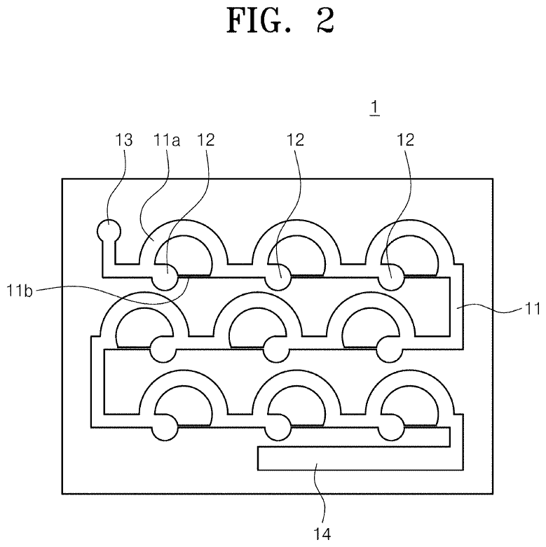

Referring to FIG. 1, in vitro fertilization may be induced by circulating a solution 30, in which sperms are mixed with Xenopus laevis spawns located in the passage 11 in the chip 10 in the Xenopus laevis chip 10 through a peristaltic pump 20. The Xenopus laevis chip 10 includes the plurality of recesses 12, and may function to trap organisms, on which spawning and in vitro fertilization of Xenopus laevis have been completed. A detailed structure of the Xenopus laevis chip 10 will be described in detail in FIG. 2. The organisms, on which the in vitro fertilization has been completed, may be tadpoles.

The pump 20 according to the inventive concept may perform a control such that the sample flows in any one of a first direction or a second direction. The pump 20 may adjust a direction and a velocity of the flows of the sample. According to an example, the pump 20 is used to induce the flows of the solution 30, and according to an example, the pump 20 may be a peristaltic pump or a syringe pump. According to an embodiment, when the peristaltic pump 20 is used, a fluid may be made to circulate by causing sperms, therapeutic substances, or biomolecules to continuously flow at a constant velocity for several hours or several days.

According to an embodiment, the sample supplied to the chip 10 by using the pump 20 may be any one of the solution 30 containing spawns, the solution 30 containing sperms that may sterilize the spawns, or a pharmaceutical sample that may evaluate toxicity. According to an example, the sample may be a sample that is related to development of a Xenopus laevis frog.

According to an embodiment, the Xenopus laevis chip 10 included in the toxicity evaluation system 1 according to FIG. 1 may control physical movements of eggs and embryos in a single entity level.

According to the inventive concept, an effect and a toxicological operation of a material may be evaluated by trapping an egg at one trapping site, that is, in the recess 12, inducing egg fertilization by circulating sperms through a fluid circulating system, and circulating therapeutic substances or biomolecules. According to an example, operations, expressive types of toxics, and mechanism of various medicines, such as therapeutic substances or biomolecules, in particular, NSAIDs may be known. Furthermore, a direction of the flows of the fluid may be adjusted by using the pump 20. According to an example, when the sample flows in a forward direction, a single entity may be trapped, and when the sample flows in a reverse direction, the trapped entities may be extracted out of the 3D chip 10.

FIG. 2 is a view illustrating the chip 10 according to an embodiment of the inventive concept.

Referring to FIG. 2, the chip 10 may be configured to include the passage 11 and the recesses 12. According to an example, each of the plurality of recesses 12 may have a size, by which one spawn included in the sample is trapped. The spawn may be an egg. According to an example, an inlet port 13 and an outlet port 14 may be formed at one end and an opposite end of the passage 11. The inlet port 13 and the outlet port 14 may be connected to the pump 20 to function as an introduction port 13 or a discharge port 14 of the sample.

Referring to FIG. 2, the passage 11 may be a combination of a first passage 11a having a first width and a second passage 11b having a second width. The first width may be larger than the second width. The first passage 11a may be a passage, in which the sample mainly flows. The second passage 11b may connect the first passage 11a and the recess 12. The second passage 11b may connect the first passage 11a and the recess 12, and may function to add a pressure such that the sample flows better as it has the second width that is smaller than the first width.

According to an example, the second passage 11b may be omitted.

According to an example, the first width may have a size, by which the object included in the sample may flow, and the second width may have a size, by which the object included in the sample is prevented from flowing. The recess 12 may be provided in a direction that faces the outlet port 14 from the inlet port 13.

Referring to FIG. 2, because the recess 12 is provided at a front part in the direction that faces the outlet port 14 from the inlet port 13, it may be a site, at which the spawn included in the sample is trapped. Because the recess 12 has a size, by which one spawn may be trapped, the spawns may be trapped in the recesses to be easily observed individually when the sample continuously flows through the plurality of recesses 12 disposed on the passage 11. Conventionally, because the chip itself is not used, the spawns gather together so that it is difficult to directly observe a development process. The inventive concept suggests a chip structure that may solve the problem.

According to an example, the Xenopus laevis chip 10 according to the inventive concept may have four functions. A single spawn may be trapped (single egg trapping), external fertilization (in vitro fertilization) may be possible, and development and escaping of a tadpole may be performed.

The drawing of the Xenopus laevis chip 10 disclosed in FIG. 2 may be merely an example, and another shape may be provided as long as it has a shape that satisfies the described condition.

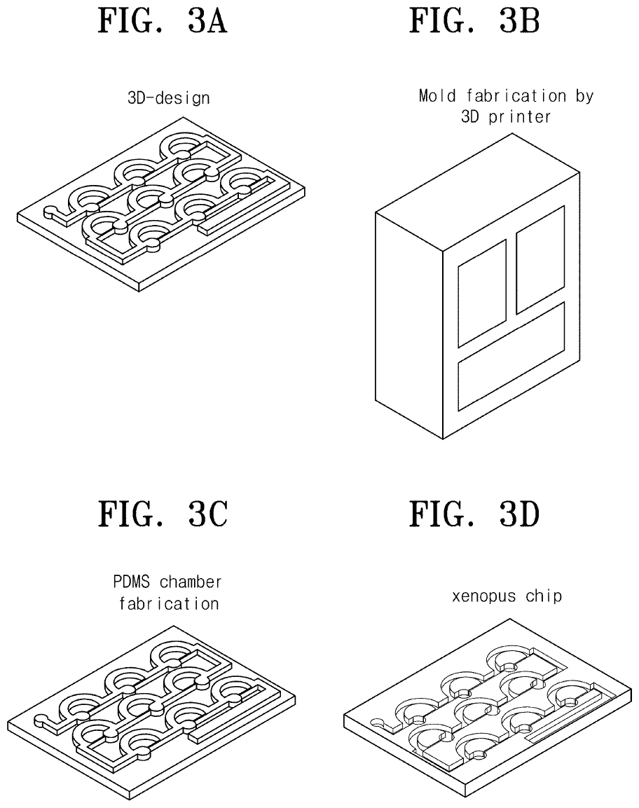

FIG. 3 is a view illustrating a process of manufacturing the chip 10 according to FIG. 2.

According to FIG. 3A, implementation of a 3D design is illustrated. According to FIG. 3B, an example of outputting a mold by using a 3D printer is disclosed. According to FIG. 3C, manufacturing of a PDMS chamber using the mold is illustrated. According to FIG. 3D, it may be identified that the Xenopus laevis chip 10 is manufactured.

Referring to FIG. 3, a 3D structure is drawn by using software for a 3D design, and a 3D mold may be output by using 3D printer equipment. A polydimethlysiloxane (PDMS) mixture may be poured into the output 3D mold, may be solidified at a temperature of about 70° C. to 80° C., and may be separated. Thereafter, the Xenopus laevis chip 10 may be manufactured by attaching another flat PDMS layer or a slide after the inlet port 13 and the outlet port 14, through which the solution 30 may be injected into the PDMS layer, are punched with a biopsy punch. That is, according to the inventive concept, the mold maybe manufactured through the 3D printer, and the Xenopus laevis chip 10 may be manufactured of polydimethlysiloxane (PDMS) through soft lithography.

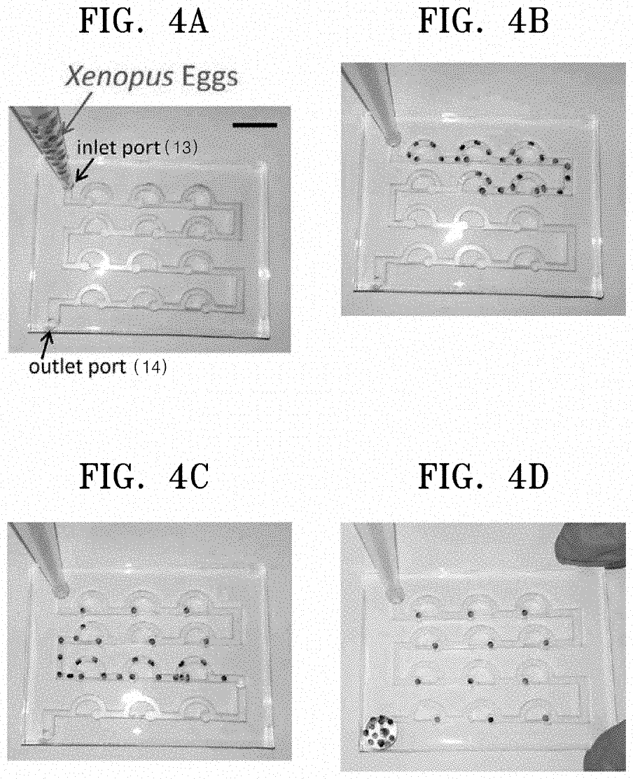

FIG. 4 is a view illustrating that spawns are trapped by using the chip 10 according to FIG. 3.

According to an embodiment, when the solution 30 having eggs is injected in the forward direction, one egg may be trapped at a trapping site, that is, in the recess 12. When one trapping site, that is, the recess 12 is filled, the remaining entities may move to the next trapping site, that is, another recess 12 through a bypass, that is, the first passage 11a. Furthermore, when the fluid flows in the reverse direction, the entities trapped in the recesses 12 may be extracted out of the chip 10.

In more detail, the Xenopus laevis chip 10 may have the inlet port 13, through which the solution 30 may be injected, and the outlet port 14, through which the solution 30 may be extracted, and may include the plurality of recesses 12 that may be trapping sites, the first passage 11a, and the second passage 11b. When the Xenopus laevis spawns are collected in a pipette tip and are injected through the inlet port 13, the spawns move along the first passage 11a. When one spawn is trapped in the recess 12 while the sample containing the spawns flow along the first passage 11a, the remaining spawns return through the first passage 11a. By repeating the process, the spawns are trapped in the plurality of recesses 12 sequentially one by one, and after the spawns are trapped in all of the plurality of recesses 12, the remaining spawns are extracted toward the outlet port 14.

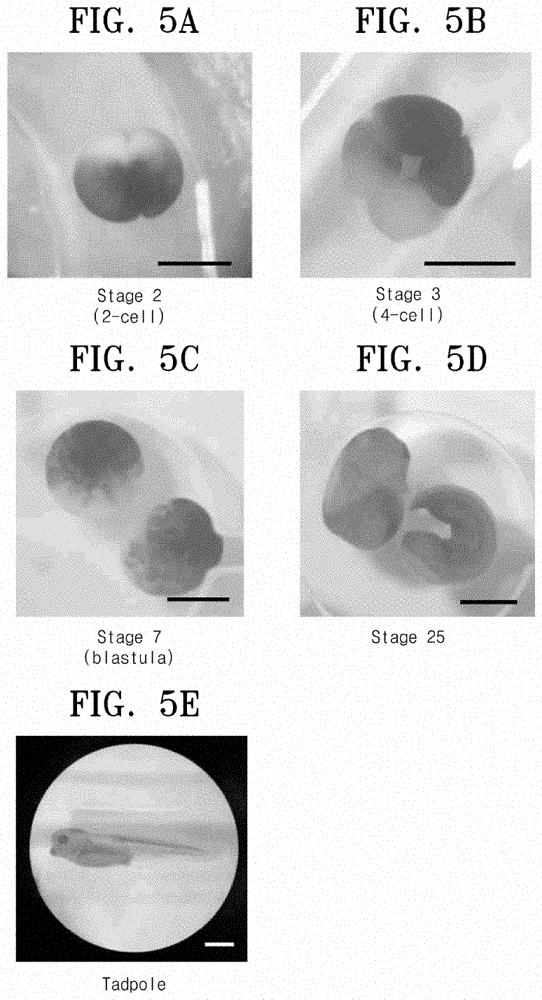

FIG. 5 is a view illustrating that an embryo is sequentially grown by using the chip 10 according to the inventive concept. In more detail, it is a view illustrating a development process of a spawn, on which in vitro fertilization has been performed in the Xenopus laevis chip 10.

FIG. 5A illustrates a step, in which cell cleavages are observed in a spawn, on which the in vitro fertilization has been performed in the Xenopus laevis chip 10. A two cell state of FIG. 5A corresponds to stage 2 of the development of a Xenopus laevis. A four cell state of FIG. 5B corresponds to stage 3 of the development of the Xenopus laevis. A blastula state of FIG. 5C corresponds to stage 7 of the development of a Xenopus laevis. FIG. 5D illustrates a picture corresponding to stage 25. According to FIG. 5E, a tadpole is observed when development of about 50 hours or more is progressed.

When movement of the tadpole that was in vitro fertilized in the Xenopus laevis chip 10 and was grown up is observed, it may be observed heat beats, tailing, and swimming.

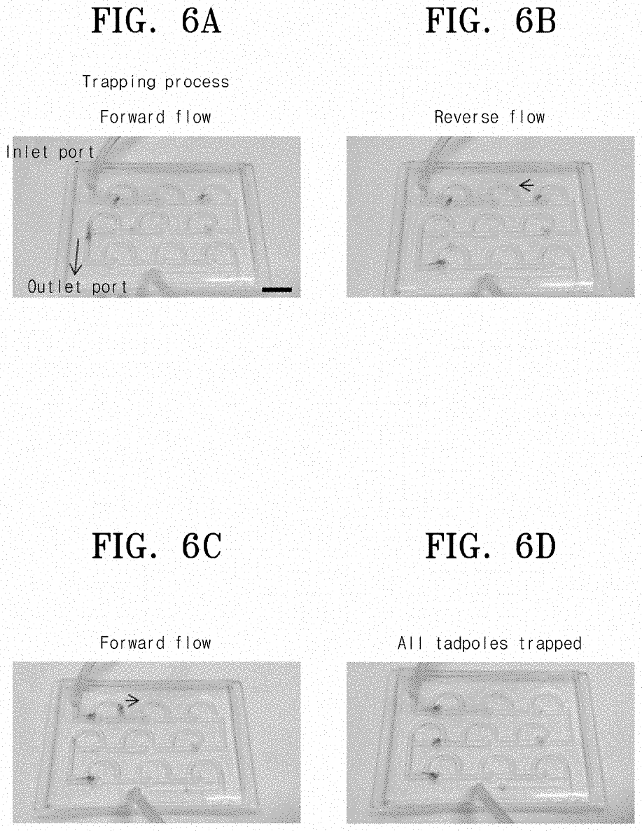

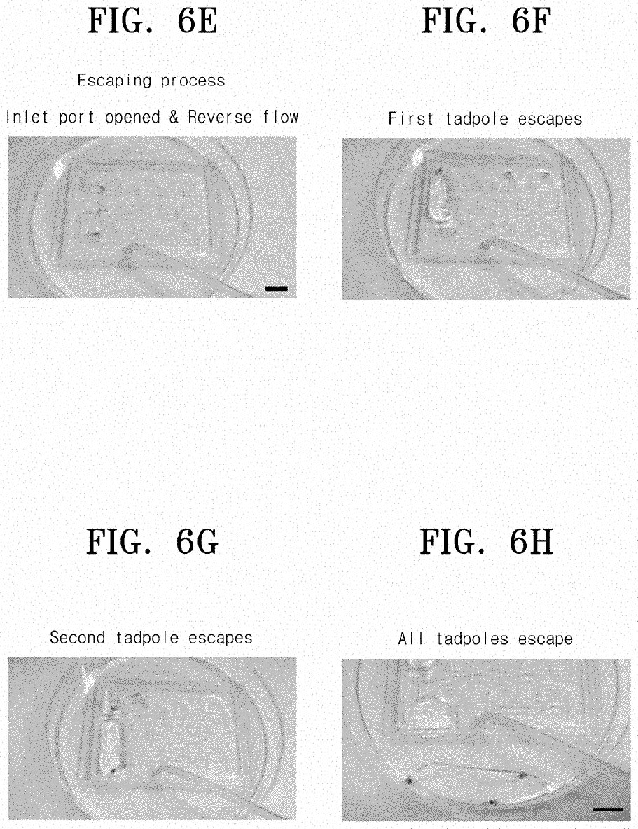

FIG. 6 is a view illustrating that trapping or escaping of the tadpole may be adjusted by adjusting a direction of the pump 20.

A structure of the Xenopus laevis chip 10 has an asymmetrical shape. The flows of the solution 30 may be adjusted to a forward or reverse direction through the peristaltic pump 20, and thus has different functions. FIGS. 6A to 6D illustrate that a trapping process of the tadpole may be induced when the sample flows in the forward direction. FIGS. 6E to 6H illustrate that an escaping process of the tadpole may be induced when water flows in the reverse direction.

Referring to FIG. 6A, three tadpoles are in the Xenopus laevis chip 10, and two of them are at the trapping sites, that is, in the recesses 12 and one of them is in the first passage 11a. The solution 30 may flow to the recess 12 as in FIG. 6B by causing the solution 30 to flow in the forward direction. Next, as in FIG. 6B, when the solution 30 flows in the reverse direction, one of the three tadpoles may be extracted from the recess 12. As in FIG. 6C, it may be observed that the tadpole extracted from the recess 12 may be located in the first passage 11a, and in the process, the remaining two tadpoles may be resistant to the reverse flows. To move all the tadpoles to the recesses 12, as in FIGS. 6C and 6D, the solution 30 may be made to flow in the forward direction.

That is, the tadpoles may be moved to desired recesses 12 by adjusting the flows of the solution in the forward direction and the reverse direction. In particular, the heads of the tadpoles are positioned toward the recesses 12 and the tails of the tadpoles are positioned toward the passage 11 by inducing the repeated forward and reverse flows, and thus the directions, in which the tadpoles are positioned, may be adjusted. Through this, a toxicity evaluation may be possible in the same condition.

According to FIGS. 6E to 6H, a process of causing the tadpole to escape out of the chip 10 to obtain the tadpole that has been externally fertilized, developed, and grown up in the chip 10 is illustrated.

As in FIG. 6E, a tube connected to the inlet port 13 of the chip 10 is extracted and stronger reverse flows are induced. As in FIG. 6F, it may be observed that the first tadpole is extracted. It may be observed in FIG. 6G that the second tadpole is extracted. As in FIG. 6H, it may be observed that all of the three tadpoles are extracted. The extracted tadpoles may be contained in a Petri dish. Through this, it is possible to secure the three tadpoles that have been in vitro-fertilized, developed, and grown up in the chip 10.

The tadpoles may be located at desired trapping sites, that is, in the recesses 12 by carefully adjusting the flows of the solution 30 in the forward or reverse direction through the peristaltic pump 20 to control movements of the tadpoles.

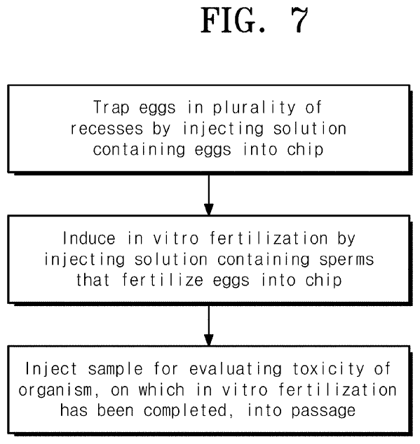

FIG. 7 is a flowchart illustrating a method for a toxicological evaluation according to an example of the inventive concept.

Referring to FIG. 7, a method for performing a toxicity evaluation by using the toxicity evaluation system 1 is disclosed. First, the 3D chip 10 may be designed and manufactured. This is illustrated in FIGS. 2 and 3. The method may include an operation of trapping the spawns in the plurality of recesses 12 by injecting the solution 30 containing the spawns into the chip 10, and an operation of inducing in vitro fertilization by injecting the solution 30 containing the sperms that fertilize the spawns into the chip 10. In more detail, ovulation may be induced through injection of HCG into the Xenopus laevis chip 10, and it may be injected into the 3D chip 10 after the spawns are squeezed. Thereafter, in a process of injecting the spawns into the 3D chip 10, single entities may be physically fixed to the recesses 12. Then, based on the peristaltic pump 20, the fluid circulating system 1 that may inject sperms into the 3D chip 10 may be constituted. Thereafter, in vitro fertilization may be induced by circulating the sperms for several hours or several days. Growth of cleavages or tadpoles may be observed through this, or growth of cleavages or tadpoles may be observed while medicines are injected.

Thereafter, an operation of injecting a sample for evaluating a toxicity of a living body, on which the in vitro fertilization has been completed, into the passage 11 may be included. According to an example, in addition to the toxicity evaluation of an organism, on which the in vitro fertilization has been completed, the toxicity evaluation may become possible by injecting the sample for the toxicity evaluation even in the development process.

Thereafter, when the growth of the tadpoles are finished, the entities may be extracted out of the 3D chip 10. Thereafter, embryogenesis may be studied through a molecular-biological analysis including an analysis of genes.

According to an example, by causing the solution to flow in a first direction by adjusting the pump 20, the organism, on which the in vitro fertilization has been completed, may be trapped in the recess 12. According to another example, by causing the solution to flow in a second direction by adjusting the pump 20, the organisms, on which the in vitro fertilization has been completed, may be caused to escape.

That is, according to the inventive concept, external fertilization may be observed, and may be utilized for pharmaceutical toxicology. Furthermore, side effects that may be caused as drugs are mixed with the solution 30 and are caused to flow in the external fertilization, development, and growth process in the chip 10.

Furthermore, the inventive concept may circulate the solution 30, in which the sperms are mixed for external fertilization, by using the pump 20. Alternatively, the solution for cultivating Xenopus laevis may be circulated, or drugs may be mixed therewith to be circulated. Furthermore, the chip 10 may be utilized for the study of embryogenesis.

According to the inventive concept, after external fertilization of embryos, a toxicity, by which therapeutic substances or biomolecules influence development of a vertebrate, may be evaluated.

The effects of the inventive concept are not limited to the above-mentioned effects, and the unmentioned effects can be clearly understood by those skilled in the art to which the inventive concept pertains from the specification and the accompanying drawings.

It is noted that the above embodiments are suggested for understanding of the inventive concept and do not limit the scope of the inventive concept, and various modifiable embodiments also fall within the scope of the inventive concept. It should be understood that the technical protection range of the inventive concept has to be determined by the technical spirit of the claims, and the technical protection range of the inventive concept is not limited to the lexical meaning of the claims but reaches even to the equivalent inventions.

What is claimed is:

1. A toxicity evaluation system comprising:
a chip including:
a passage, in which a sample flows; and
a plurality of recesses provided on the passage, and configured to trap objects included in the sample; and
a pump configured to inject the sample into the chip.

2. The toxicity evaluation system of claim 1, wherein each of the plurality of recesses has a size, by which one spawn included in the sample is trapped.

3. The toxicity evaluation system of claim 2, wherein an inlet port and an outlet port are formed at one end and an opposite end of the passage.

4. The toxicity evaluation system of claim 3, wherein the passage is a combination of a first passage having a first width and a second passage having a second width,
wherein the first width has a size, by which the objects included in the sample flow, and
wherein the second width has a size, by which the objects included in the sample is prevented from flowing.

5. The toxicity evaluation system of claim 4, wherein the recesses are provided in a direction that faces the outlet port from the inlet port.

6. The toxicity evaluation system of claim 2, wherein the pump causes the sample to flow in any one of the first direction or the second direction.

7. The toxicity evaluation system of claim 6, wherein the sample is any one of a solution containing the spawns, a solution containing sperms that fertilize the spawns, or a pharmaceutical sample, by which a toxicity is evaluated.

8. A method for performing a toxicological evaluation by using the toxicity evaluation system of claim 2, the method comprising:
trapping the spawns in the plurality of recesses by injecting the solution containing the spawns into the chip; and
inducing in vitro fertilization by injecting the solution containing the sperms that fertilize the spawns into the chip.

9. The method of claim 8, further comprising:
injecting a sample for evaluating a toxicity of an organism, on which the in vitro fertilization has been completed, into the passage.

10. The method of claim 9, further comprising:
trapping the organism, on which the in vitro fertilization has been completed, by the recesses, by adjusting the pump such that the sample flows in a first direction.

11. The method of claim 9, further comprising:
causing the organism, on which the in vitro fertilization has been completed, to escape, by adjusting the pump such that the sample flows in a second direction.
